(19) United States
(12) Patent Application Publication
(10) Pub. No.: US 2015/0272863 A1
(43) Pub. Date: Oct. 1, 2015

(54) H3 ANTAGONIST BY-PRODUCT, ITS MANUFACTURE, AND METHOD OF USING BY-PRODUCT TO REDUCE PIGMENTATION IN SKIN

(71) Applicant: Active Concepts, LLC, Lincolnton, NC (US)

(72) Inventors: Durant Sholz, Nanuet, NY (US); Erica Segura, Huntersville, NC (US); Meghan Darley, Belmont, NC (US); Maureen Danaher, Charlotte, NC (US)

(21) Appl. No.: 14/666,864

(22) Filed: Mar. 24, 2015

Related U.S. Application Data
(60) Provisional application No. 61/970,007, filed on Mar. 25, 2014.

(51) Int. Cl. A61K 8/97; A61Q 19/02

(52) U.S. Cl. CPC A61K 8/97; A61Q 19/02; A61K 2800/85; A61K 2800/782

(57) ABSTRACT

The invention relates to a method of reducing skin pigmentation. The method includes the steps of providing an H3 antagonist configured to prevent histamine from connecting to an H3 receptor, thereby preventing the H3 receptor from initiating melanin production; adding the H3 antagonist to a cosmetic product; and applying the cosmetic product to a user's skin.

### BACKGROUND OF THE INVENTION

The invention relates to the field of cosmetics, and more particularly to the use of a cosmetic product for skin lightening.

The drive for skin perfection is well known in Asian communities as Asian woman strive for porcelain-like skin. Such aesthetic perfection has also become a consumer standard in western markets. Uneven skin tone is often associated with age or damaged skin. As a result, consumers are routinely looking for products that can provide them with younger looking skin.

One way to obtain younger looking skin is to use a skin lightening product. As a result, such products have become a staple in the cosmetic industry. Skin lightening products inhibit melanin production, which causes pigmentation or dark spots in the skin, by preventing receptors from interacting with histamine. Until now, it was believed that only H1 and H2 antagonists could be used to reduce or eliminate pigmentation. However, this application shows that a product using an H3 antagonist may be used for pigmentation reduction and that a new method of producing such product may result in enhanced benefits.

### BRIEF SUMMARY OF THE INVENTION

These and other shortcomings of the prior art are addressed by the present invention, which provides a skin lightening product based on an H3 antagonist.

According to one aspect of the invention, a method of reducing skin pigmentation includes the steps of providing an H3 antagonist configured to prevent histamine from connecting to an H3 receptor, thereby preventing the H3 receptor from initiating melanin production; adding the H3 antagonist to a cosmetic product; and applying the cosmetic product to a user's skin.

According to another aspect of the invention, a method of reducing skin pigmentation includes the steps of providing an H3 antagonist configured to connect to an H3 recepter and prevent the H3 receptor from initiating melanin production, the H3 antagonist being extracted from fermented Vitis vinifera; adding the H3 antagonist to a leave-on product; and applying the leave-on product to a user's skin.

According to another aspect of the invention, a method of producing an H3 antagonist by-product configured to prevent H3 receptors from initiating melanin production includes the steps of providing plant feedstock, processing the plant feedstock, fermenting the plant feedstock using a bacteria, and extracting an H3 antagonist from the fermented plant feedstock.

### BRIEF DESCRIPTION OF THE DRAWINGS

The subject matter that is regarded as the invention may be best understood by reference to the following description taken in conjunction with the accompanying drawing figures in which:

FIGS. 1-3 show results of melanin inhibition assays for known H3 antagonists;

FIGS. 4-6 show melanin inhibition assay results for the H3 antagonist by-product of the current invention;

FIG. 7 shows a process for producing the H3 antagonist by-product of the current invention;

FIGS. 8 and 9 show tyrosinase inhibition assay results for the H3 antagonist of the current invention compared to known inhibitors;

FIG. 10 shows tyrosinase inhibition assay results of the H3 antagonist by-product of the current invention compared to individual components of the by-product;

FIG. 11 shows melanin inhibition assay results of the H3 antagonist by-product of the current invention compared to individual components of the by-product; and

FIGS. 12A-12C and 13A-13C are photos of pigmentation reduction in a subjects skin after use of the H3 antagonist by-product.

### DETAILED DESCRIPTION OF THE INVENTION

Skin pigmentation is determined by the amount of melanin in the skin. Melanin is produced by dermal melanocytes. Melanogenesis is the process that leads to the formation of melanin. It is initiated in melanosomes, the special organelles of melanocytes, with the oxidation of L-tyrosine to L-dopa (L-3, 4-dihydroxyphenylalanine) and then to dopaquinone, which is catalyzed by tyrosinase. Overproduction of melanin can cause hyperpigmentation leading to melisma, freckles, age-spots, and liver spots.

It is well known that H1, H2, and H3 are receptors in inflammatory pathways of the human body. It has been shown that H1 and H2 antagonist by-products are suitable for reducing pigmentation in the skin, i.e., age spots or sun damage, by blocking the H1 and H2 pathways. However, until now, it has been assumed that H3 antagonist by-products would not be suitable for the same use.

Through experimentation, it has been found that H3 antagonists such as Impentamine dihydrobromide; lodophenpropit; (R)-(-)-a-Methylhistamine dihydrobromide; Conessine; and Imetit dihydrobromide are in fact suitable for use as a by-product in cosmetics to reduce or eliminate pigmentation in a users skin.

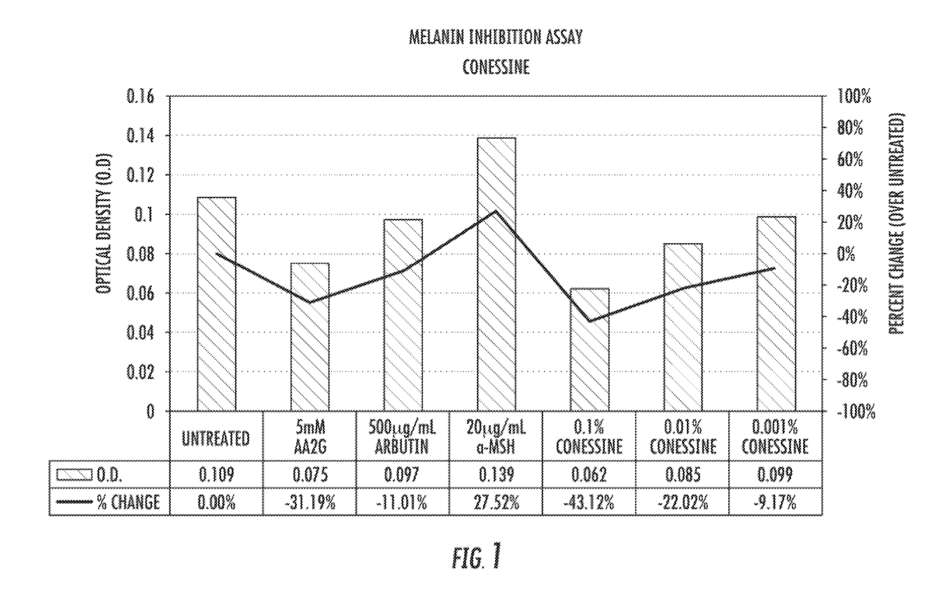

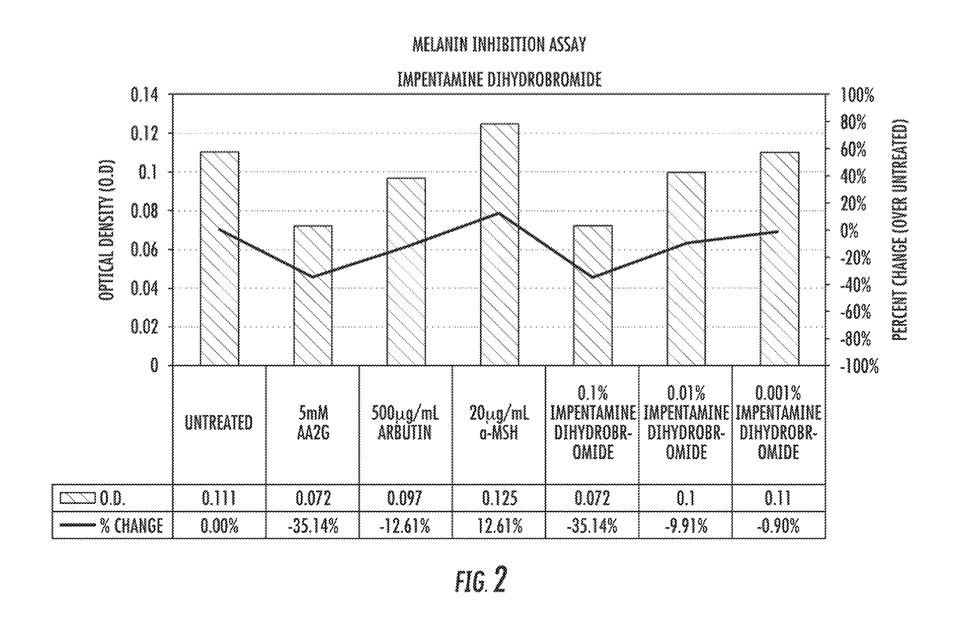

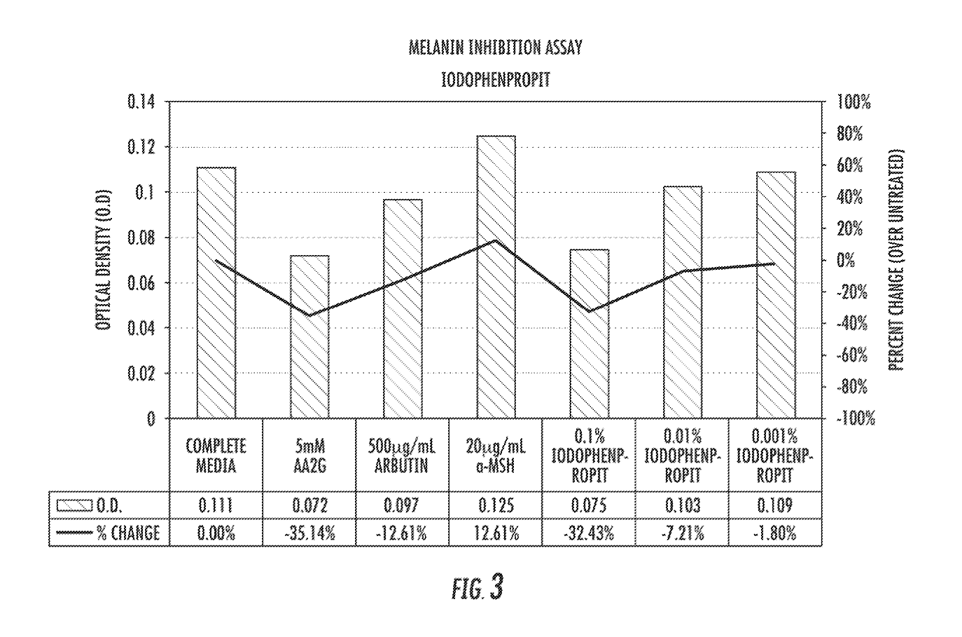

Melanin inhibition studies were conducted using several H3 antagonists to assess their ability to inhibit melanin synthesis. FIGS. 1-3 show the results for Conessine, Impentamine dihydrobromide, and lodophenpropit.

The assays utilized mouse melanoma cells (B16F10 cell line) which are very efficient at producing melanin. Various inhibitors and enhancers of melanin synthesis were added to the cultured cells along with experimental samples. The melanin present in the cells was extracted and had an absorbance at 400 nm, which can be quantitated through optical density measurements. The greater the inhibition exhibited by the sample, the lower the optical density value due to the lack of melanin present. The results of the assays were analyzed and compared to known melanin inhibitors, such as Arbutin and ascorbic acid 2-glucoside (AA2G).

The B16F10 murine melanocytes were seeded into 24-well tissue culture plates and allowed to grow to confluency in complete DMEM. Solutions of H3 antagonists, Arbutin, alpha-Melanocyte Stimulating Hormone (a-MSH), and AA2G were prepared in Complete DMEM. Complete Dulbeco's Modified Eagle Media (DMEM) was used as the untreated control. Additionally, the individual components of the H3 antagonists, Yeast Peptide and Plant Extract, were also analyzed for melanogenic effects. Cells were treated for 72 hours.

For the inhibition assay, the media was removed from the wells and the monolayers were washed with phosphate buffered saline (PBS). NaOH was added to each well and 100 μL of each solution were added to a 96-well plate in duplicate. The plate was measured by optical density readings at 400 nm. As shown in FIGS. 1-3, each of the H3 antagonists was capable of inhibiting melanin production.

The H3 antagonist by-product of the present invention is a fermented plant extract (the terms H3 antagonist by-product and fermented plant extract are to be appreciated as referring to the same by-product). The by-product is typically used in leave-on products such as serums, lotions, cremes, etc. The by-product could be in liquid or powder form and is mixed with or added to a cosmetic company's existing product to allow a user to apply the cosmetic product to skin as usual.

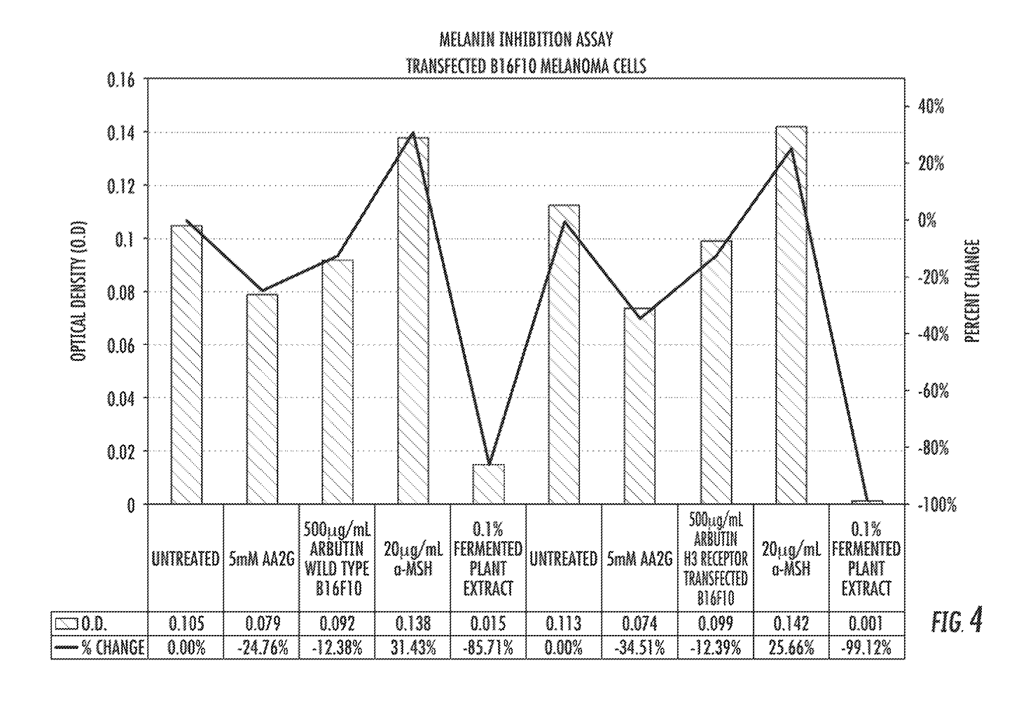

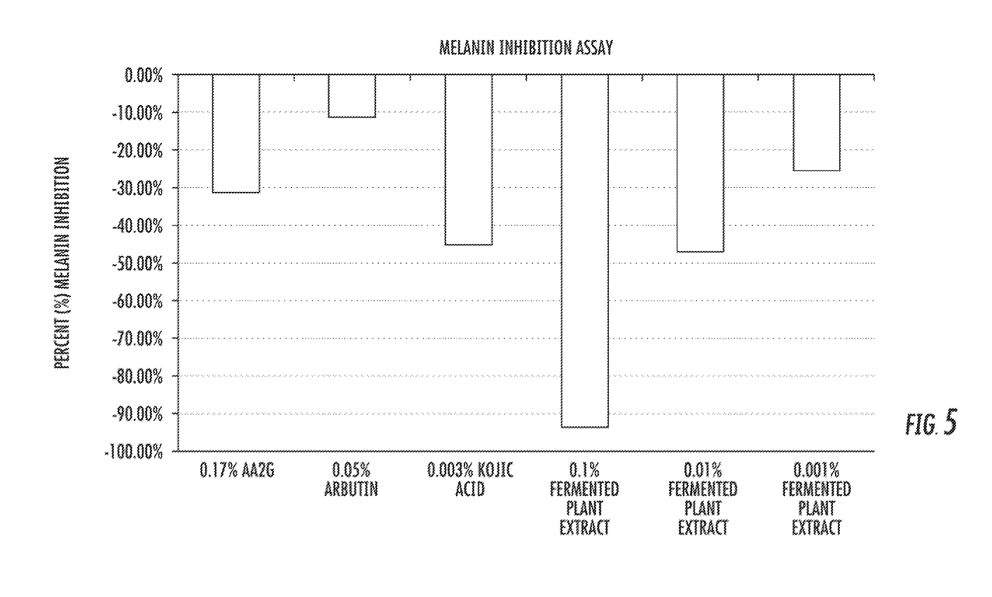

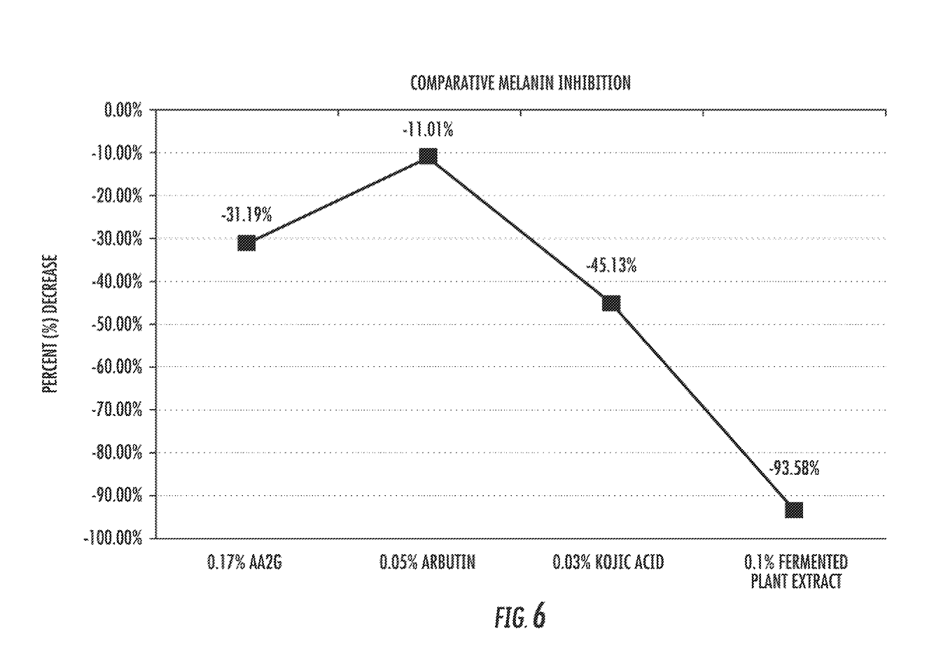

As skin is exposed to ultraviolet radiation, the inflammatory pathway releases histamine. This histamine binds with receptor binding sites and causes the production of melanin. The H3 antagonist by-product of the current invention prevents histamine from connecting to H3 receptors by connecting itself to the receptor. Because the H3 antagonist by-product does not talk to the receptor like histamine does, the H3 antagonist by-product prevents the receptor from initiating melanin production. FIG. 4 shows the results of a melanin inhibition assay comparing the H3 antagonist by-product of the current invention with AA2G, Arbutin, and α-MSH. As shown, the H3 antagonist by-product was effective as a melanin inhibitor. FIGS. 5 and 6 show additional results of the melanin inhibition assay comparing concentrations of the H3 antagonist by-product to AA2G, Arbutrin, and Kojic acid. Again, the H3 antagonist by-product was shown to be effective as a melanin inhibitor.

The H3 antagonist by-product of the current invention is produced using a fermentation process. In general, the fermentation process includes the steps of:
1. Providing feedstock;
2. Processing the feedstock (i.e., macerating, etc.);
3. Fermenting the feedstock using yeast or suitable bacteria;
4. Using a solvent to extract the antagonist and any other beneficial components/compounds; and
5. Filtering to clarify solution and remove any remaining feedstock material.

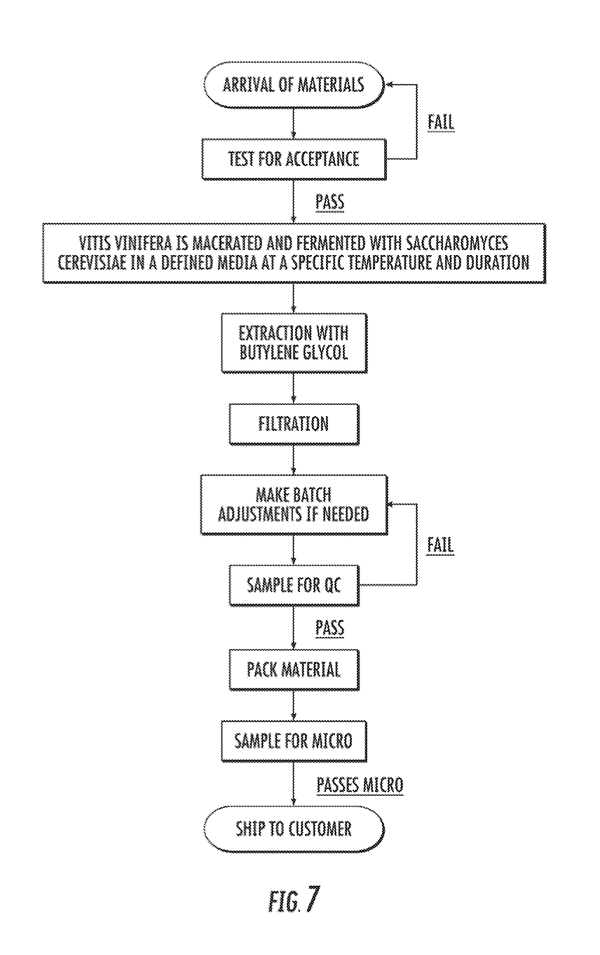

More particularly, the H3 antagonist by-product is manufactured using Vitis vinifera which is macerated and fermented with yeast. Fermentation media is comprised of ammonium sulfate, magnesium sulfate, disodium phosphate, and yeast autolysate. An antimicrobial compound or product is added and then the solution is filtered. See FIG. 7 for more detailed steps.

It was found in testing that the fermenting process produces a more potent H3 antagonist by-product. Assays for Tyrosinase Inhibition and Melanin Inhibition were performed.

### Tyrosinase

As discussed above, tyrosinase is one of the causes of hyperpigmentation, and over-production of dermal melanin pigment, leading to melasmas, freckles, age-spots, and liver spots. Tyrosinase is a key enzyme in melanin biosynthesis, involved in determining the color of mammalian skin and hair. Tyrosinase's main application is to identify new potent tyrosinase inhibitors in the cosmetic industry. Tyrosinase is a copper-containing monooxygenase that is widely distributed in nature. The enzyme catalyzes the first two reactions of melanin synthesis, the hydroxylation of L-tyrosine to 3, 4-dihydroxyphenylalanine, L-dopa, and the oxidation of L-dopa to dopaquinone. This quinine is a highly reactive compound and can polymerize spontaneously to form melanin.

A tyrosinase inhibition assay was conducted for the fermented plant extract (fermented H3 antagonist by-product) and its individual components—peptide from yeast and plant extract. The assay was based on the conversion of L-tyorosine into a dopachrome complex by tyorsinase. The dopachrome complex has an abosorbance at 490 nm and can be quantitated through optical density measurements. The greater the inhibition exhibited by the sample, the lower the optical density value due to the lack of L-tyrosine conversion.

For the inhibition assay, 10 μL of test material and controls were combined with 170 μL of 1 mM L-tyrosine and 20 μL 1000 U/mL mushroom tyrosinase in a 96-well microtitre plate. The plate was placed in a Synergy H1 reader set to 37° C. and optical density measurements were taken every 20 minutes at 490 nm.

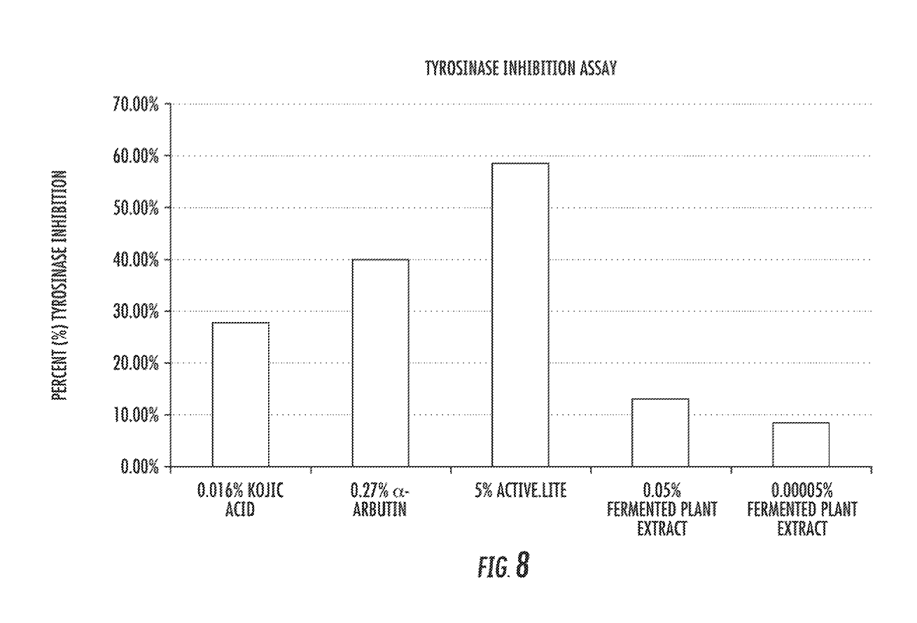

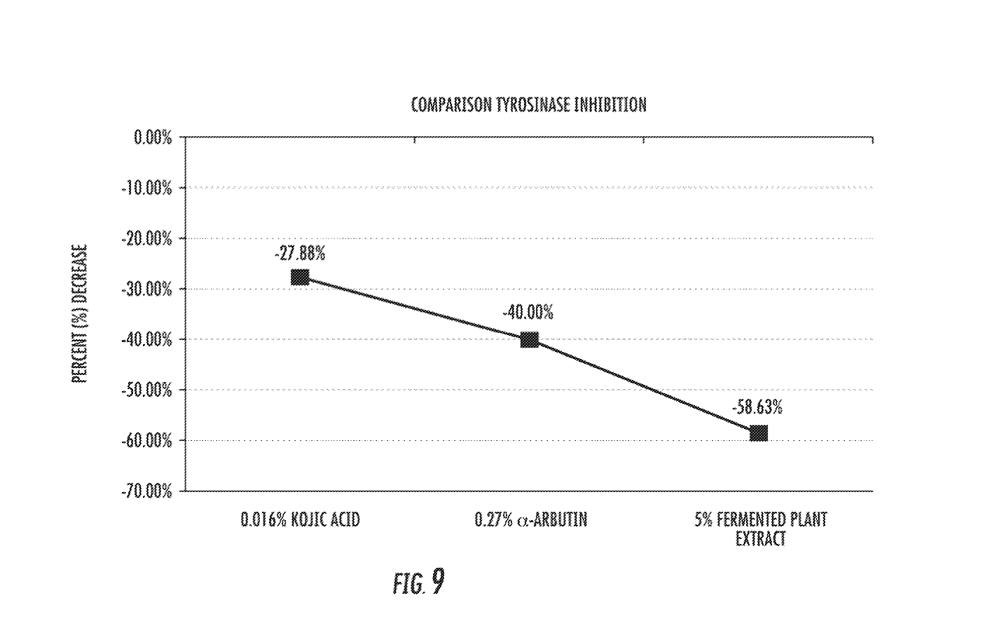

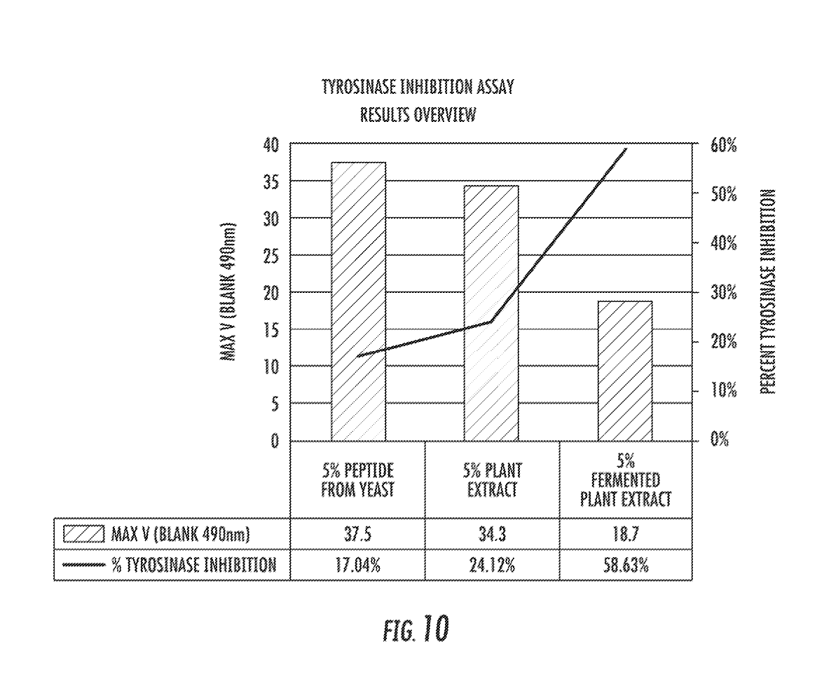

The results were analyzed and compared to known tyrosinase inhibitors, Kojic acid and Arbutrin—market standard skin lightening ingredients. See FIGS. 8 and 9. As shown, the fermented plant extract was able to inhibit tyrosinase compared to the positive control, Kojic acid. Additionally, the H3 antagonist by-product of the current invention was compared to its individual components—peptide from yeast and plant extract. As shown in FIG. 10, the fermented plant extract outperformed the peptide from yeast and non-fermented plant extract individually and exhibited a synergetic effect.

### Melanin

As discussed above, skin pigmentation is determined by the amount of melanin in the skin which is produced by dermal melanocytes. In addition to the melanin inhibition study comparing the fermented plant abstract to AA2G, Arbutin, α-MSH, and Kojic acid, a melanin inhibition study was conducted to compare the fermented plant extract with its individual components—peptide from yeast and plant extract—to assess their ability to inhibit melanin synthesis. The melanin assay was conducted using the same methodogies as discussed above with respect to H3 antagonists, in general.

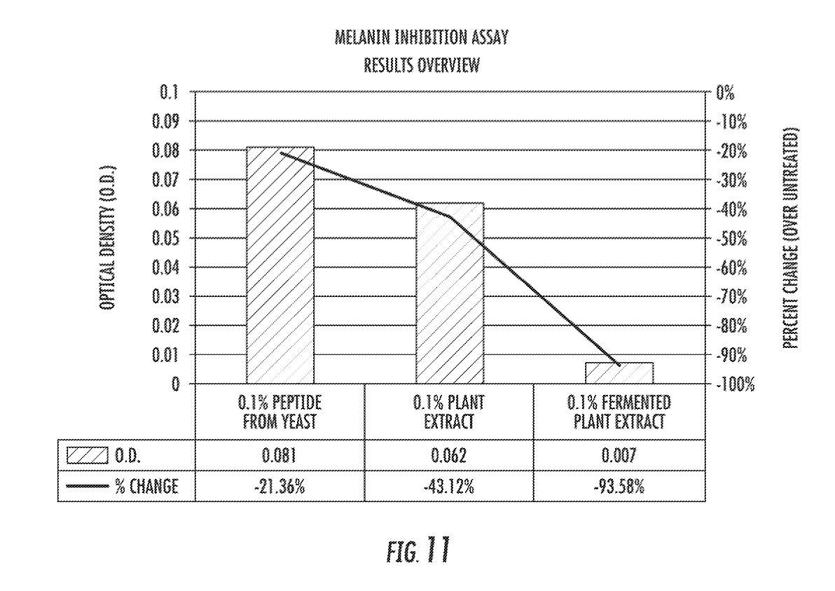

As shown in FIG. 11, the fermented plant extract was able to inhibit melanin production by 93.58% when using the product at a 0.1% concentration. The inhibition activity increased as the concentration increased. Further, the fermented plant extract outperformed the peptide from yeast and plant extract components and showed a synergistic antimelanogenic effect.

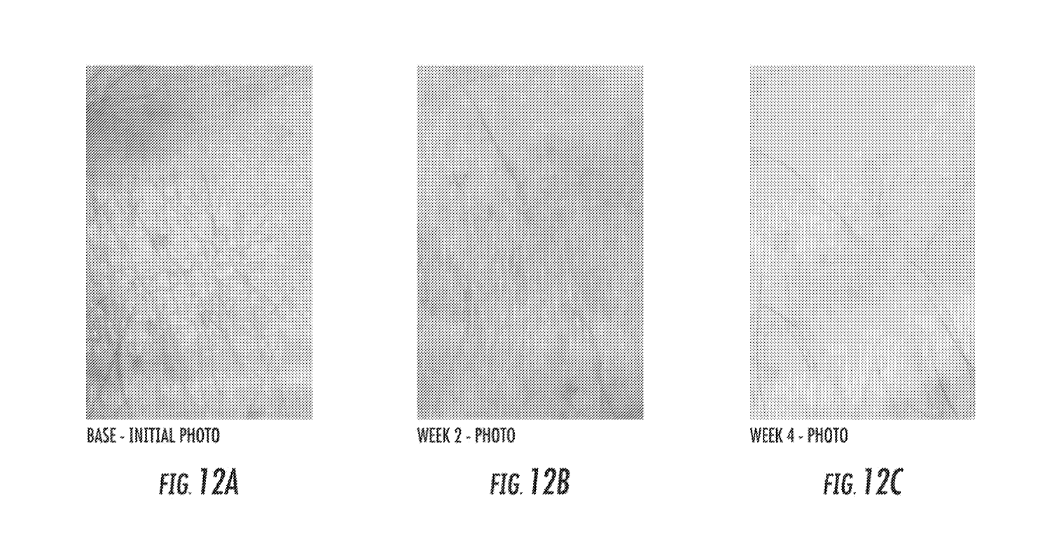

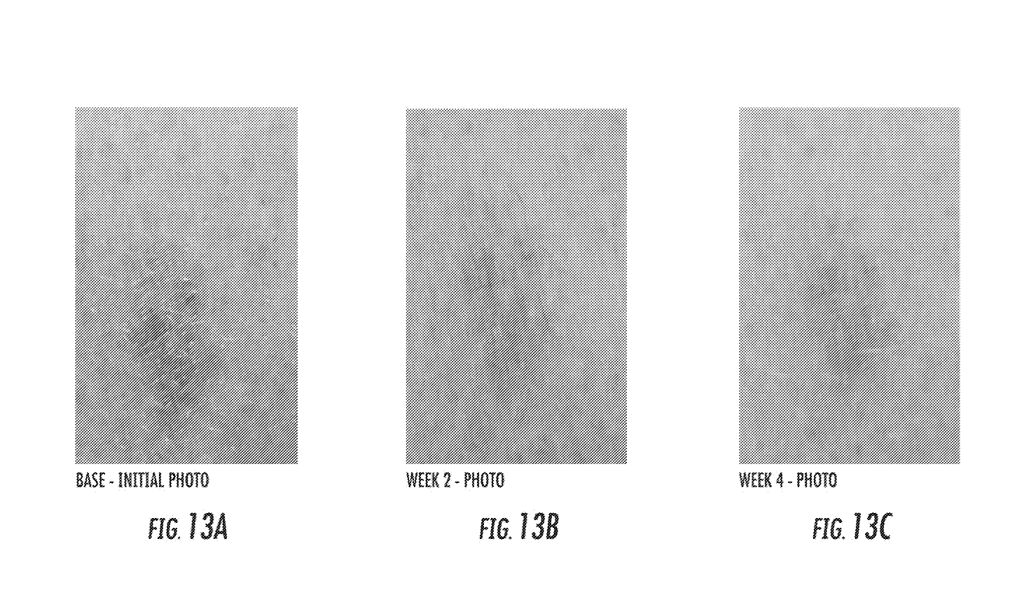

The fermented plant extract was also evaluated in its ability to lighten skin in-vivo. To test this, subjects applied a lotion containing 5.0% fermented plant extract to their selected hyperpigmentation once a day for four weeks. Photos were taken twice a week during the four week study. As shown in FIGS. 12A-12C and 13A-13C, the fermented plant extract visibly lightened dark spots on the subjects in less than 14 days of application—with even greater improvement after 4 weeks.

The foregoing has described and H3 antagonist by-product, its manufacture, and method of using by-product to reduce pigmentation in skin. While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation.

We Claim:

1. A method of reducing skin pigmentation, comprising the steps of:
(a) providing an H3 antagonist configured to prevent histamine from connecting to an H3 receptor, thereby preventing the H3 receptor from initiating melanin production;
(b) adding the H3 antagonist to a cosmetic product; and
(c) applying the cosmetic product to a user's skin.

2. The method according to claim 1, wherein the H3 antagonist is a by-product of fermented plant extract.

3. The method according to claim 1, wherein the H3 antagonist is a by-product of fermented Vitis vinifera.

4. The method according to claim 3, wherein the Vitis vinifera is fermented with yeast.

5. The method according to claim 3, wherein the Vitis vinifera is fermented with a fermentation media comprised of ammonium sulfate, magnesium sulfate, disodium phosphate, and yeast autolysate.

6. The method according to claim 1, wherein the H3 antagonist connects to the H3 receptor to prevent histamine from connecting to the H3 receptor.

7. A method of reducing skin pigmentation, comprising the steps of:
(a) providing an H3 antagonist configured to connect to an H3 recepter and prevent the H3 receptor from initiating melanin production, the H3 antagonist being extracted from fermented Vitis vinifera;
(b) adding the H3 antagonist to a leave-on product; and
(c) applying the leave-on product to a user's skin.

8. The method according to claim 7, wherein the leave-on product is selected from the group consisting essentially of serums, lotions, and cremes.

9. A method of producing an H3 antagonist by-product configured to prevent H3 receptors from initiating melanin production, comprising the steps of:
(a) providing plant feedstock;
(b) processing the plant feedstock;
(c) fermenting the plant feedstock using a bacteria; and
(d) extracting an H3 antagonist from the fermented plant feedstock.

10. The method according to claim 9, wherein the plant feedstock is Vitis vinifera.

11. The method according to claim 9, wherein the step of processing includes the step of macerating the plant feedstock.

12. The method according to claim 9, wherein the bacteria is yeast.

13. The method according to claim 9, wherein the bacteria is Saccharomyces cerevisiae.

14. The method according to claim 9, wherein the step of fermenting further includes the step of fermenting the plant feedstock in fermentation media at a pre-defined temperature for a pre-defined duration.

15. The method according to claim 14, wherein the fermentation media comprises ammonium sulfate, magnesium sulfate, disodium phosphate, and yeast autolysate.

16. The method according to claim 9, wherein the step of extracting includes the step of using butylene glycol to extract the H3 antagonist and other beneficial components or compounds and form an H3 antagonist solution.

17. The method according to claim 16, further including the step of filtering the H3 antagonist solution to clarify the solution and remove any remaining plant feedstock material from the solution.

18. The method according to claim 17, further including the step of adding an antimicrobial compound to the H3 antagonist solution prior to filtering.
